(19) United States
(12) Patent Application Publication
(10) Pub. No.: US 2022/0134182 A1
(43) Pub. Date: May 5, 2022

(54) Exercise Tracking Device For Multi-Stage Events

(71) Applicant: Wahoo Fitness L.L.C., Atlanta, GA (US)

(72) Inventors: Murray A. HUGHES, Brisbane (AU); Michael FOMIN, Brisbane (AU); Benjamin P. JOHNSTON, Brisbane (AU); Fons S.W. VAN NULAND, De Meern (NL)

(73) Assignee: Wahoo Fitness L.L.C., Atlanta, GA (US)

(21) Appl. No.: 17/086,765

(22) Filed: Nov. 2, 2020

(51) Int. Cl. A63B 24/00; H04W 4/021; H04W 4/029; G06F 1/16

(52) U.S. Cl. CPC A63B 24/0021; A63B 24/0062; A63B 24/0075; H04W 4/021; A63B 2220/12; G06F 1/163; A63B 2024/0025; A63B 2024/0068; H04W 4/029

(57) ABSTRACT

Multi-stage sporting events, such as triathlons, can present particular challenges regarding collection and processing of exercise data. For example, the general length of certain multi-stage sporting events can be problematic regarding battery life for conventional tracking devices, particularly if such devices include a relatively high quantity of sensors and/or sensors that consume particularly high amounts of power during use. This disclosure presents power management functionality to address this challenge. Multi-stage sporting events can also present challenges regarding the need to switch between different sets of sensors to measure particular parameters for a given activity. This disclosure present functionality for automatic touchless transitioning between stages of an event.

### TECHNICAL FIELD

Aspects of the present disclosure involve collection and processing of exercise data and, more specifically, collection and processing of exercise data for multi-stage sporting events using a wearable exercise tracking device that include techniques for power management and automatic detection of stage transitions.

### BACKGROUND

This section provides background information related to the present disclosure which is not necessarily prior art.

The use of portable exercise tracking devices has become increasingly common in recent years. Such devices allow users to collect and store data collected during various types of physical activity. The collected exercise data can then be processed and displayed to a user to inform the user of his or her current performance. Exercise data can also be collected and analyzed over an extended period of time such that the user can monitor his or her progress toward certain milestones, modify or optimize training routines, or otherwise inform long-term exercise plans.

Multi-stage sporting events, such as triathlons, can present particular challenges regarding collection and processing of exercise data. For example, the general length of certain multi-stage sporting events can be problematic regarding battery life for conventional tracking devices, particularly if such devices include a relatively high quantity of sensors and/or sensors that consume particularly high amounts of power during use. Multi-stage sporting events in which stages include different activities (e.g., swimming, cycling, running, etc.) can also present challenges regarding the need to switch between different sets of sensors to measure particular parameters for a given activity.

With these thoughts in mind among others, aspects of the training device disclosed herein were conceived.

### SUMMARY

This section provides a general summary of the disclosure, and is not a comprehensive disclosure of its full scope or all of its features.

In one aspect of the disclosure, a computing device is presented for use in collecting user activity data. The computing device includes: a first sensor, a processor communicatively coupled to the processor; and a memory communicatively coupled to the processor. The memory includes instructions that, when executed by the processor, cause the processor to: operate the computing device in a first mode in which first sensor data is collected using the first sensor and stored within the memory of the computing device; as well as communicatively couple the computing device with a second computing device, where the second computing device includes a second sensor for use in collecting second sensor data. The instructions further cause the processor to operate the computing device in a second mode. When operating in the second mode, the processor: operates the first sensor in a reduced power consumption mode; collects the second sensor data based at least in part from data received from the sensor of the secondary device; and stores the second sensor data in the memory.

In an example embodiment, the instructions cause the processor to transition the computing device from the first mode to the second mode in response to at least one of the computing device communicatively coupling with the second computing device, sensor data from one or more sensors of the computing device meeting a threshold, the first computing device entering a geographic area, or a change in a movement pattern of the computing device.

In some embodiments, the instructions further cause the processor to: communicatively decouple the computing device from the second computing device; and operate the computing device in a third mode. When operating in the third mode, the processor collects third sensor data using the first sensor; and stores the third sensor data in the memory.

In some embodiments, the computing device is a wearable fitness tracking device and the second computing device is a cycling computer.

In another aspect of this disclosure, a method is presented for changing operational modes of an exercise tracking device. The method includes: obtaining, at an exercise tracking device operating in a first mode, location data for the exercise tracking device; comparing the location data to geographic data stored in a memory of the exercise tracking device, the geographic data defining a geofence; and in response to determining the exercise tracking device has crossed the geofence, transitioning the computing device from the first mode of operation to a second mode of operation.

In yet another aspect of this disclosure, a method is presented for tracking exercise data for a multi-stage activity. The method includes: collecting, using a wearable computing device, a plurality of sensor data streams, automatically identifying a plurality of transitions between stages of the multi-stage activity; and in response to identifying each of the plurality of transitions, automatically changing the computing device between operational modes, where each operational mode corresponding to a respective stage of the multi-stage event. The wearable computing device records exercise-related data corresponding to the plurality of sensor data streams during each of the operational modes, and, in a subset of the operational modes, the wearable computing device is communicatively coupled to a secondary device and receives at least one of the sensor data streams from the secondary device.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

FIG. 1A is an isometric rendering of an example exercise tracking device in accordance with the present disclosure.

FIG. 1B is a rendering of an example supplemental computing device for use with the exercise tracking device of FIG. 1A.

FIG. 2 is a graphical illustration of stages of a triathlon or similar multi-stage event;

FIG. 3 is a block diagram illustrating an example operational environment according to the present disclosure;

FIG. 4 is a schematic illustration of an example exercise tracking device according to the present disclosure;

FIG. 5A-5C are block diagrams illustrating various operational modes of exercise tracking devices in accordance with the present disclosure;

FIG. 6 is a flow chart illustrating an example method of transitioning between stages of a multi-stage event;

FIG. 7 is a flow chart illustrating an example power management method for an exercise tracking device; and

FIG. 8 is a flow chart illustrating an example method for transitioning between operational modes based on a transition across a geofence.

Corresponding reference numerals indicate corresponding parts throughout the several views of the drawings.

### DETAILED DESCRIPTION

Example embodiments will now be described more fully with reference to the accompanying drawings.

Aspects of the present disclosure involve an exercise tracking device and, in particular, an exercise tracking device for use in multi-stage events, such as a triathlon. Although other features are discussed, the systems and methods described herein are particularly directed to addressing issues regarding power consumption and power management, and facilitating automatic device configurations while transitioning between stages of an event.

With respect to power and power management, triathlons and similar events can be particularly challenging due to their length and the need to incorporate a relatively broad set of sensors to accurately track activity in each stage. These issues are exacerbated when increasingly sophisticated and power hungry sensors are implemented. Accordingly, a trade-off between increased battery life and quantity and accuracy of data is often required.

To address these and other issues, implementations of the present disclosure include power management functionality in which an exercise tracking device, such as a wrist-mounted smart watch style tracker, pairs with and offloads at least some data collection functionality to a secondary device, such as a cycling computer. For example, the exercise tracking device may include a global positioning system (GPS) sensor or similar high power consumption sensor. The GPS sensor may be used to determine a location of the exercise tracking device during each of a swim stage and a running stage of a triathlon. However, during a cycling stage, the exercise tracking device may pair with a cycling computer that includes its own GPS sensor and the exercise tracking device may instead receive location information from the GPS sensor of the cycling computer. As a result, the exercise tracking device may disable or otherwise operate its own GPS sensor in a reduced power mode (including turning the GPS sensor off) to conserver battery life.

Similar power conservation techniques may be applied to other sensors and components of the exercise tracking device. For example, in one implementation, each of the exercise tracking device and the secondary device may include a display. When paired, the exercise tracking device may forward data to the secondary device to be shown on the display of the secondary device. By doing so, the exercise tracking device may turn off or otherwise operate its display in a reduced power mode, thereby conserving power. Besides saving power, display information is moved to the cycling computer allowing the athlete to better view cycling metrics and overall metrics related to their performance and the event, and not have to operate or view a separate wrist mounted device.

As previously noted, aspects of the present disclosure are also directed to automatically transitioning between stages of an event. In general, stages of an event may require different sensors to collect and record data relevant to each stage. Similarly, athletes are often interested in data for individual stages, requiring different functions (e.g., timers, pacing metrics, etc.) to be selectively activated and deactivated for each stage. To the extent such changes require manual intervention by the athlete, doing so can be a distraction from the event and, if missed, can hamper the athlete's ability to accurately track their progress during the event.

To address this issue, aspects of the present disclosure are directed to automatically identifying transitions between stages of an event using an exercise tracking device and, in response to identifying such transitions, modifying an operational mode of the exercise tracking device to reflect the new stage. Such transitions are identified based on, among other things, sensor data collected by the exercise tracking device, identifying when the exercise tracking device pairs with or connects to another device, and other similar events. In at least one specific implementation of the present disclosure, transitions between stages are identified based, at least in part, on the exercise tracking device moving across a geofence stored in a memory of the exercise tracking device. Various approaches to generating and storing the geofence data are also provided herein. Transitions between stages are critical for competitive athletes, and the transition area can often be chaotic and stressful. Not having to manually operate fitness tracker and instead focus on the transition, saves time and helps the athlete focus on the transition and not operating an exercise tracker.

In certain implementations, the exercise tracking device may operate in conjunction with one or more secondary computing devices, such as a cycling computer, and may selectively communicate with the secondary computing devices based on the operational mode of the exercise tracking device. In general, however, even when connected to one or more external devices, the exercise tracking device acts as the “master” of the event data. In other words, the exercise tracking device collects and records all exercise data in a memory of the exercise tacking device and generally performs any processing and analysis of the collected data for purposes of displaying information to a user of the device.

These and other aspects of the present disclosure are now provided in further detail.

Overview of an Example Multi-Stage Event

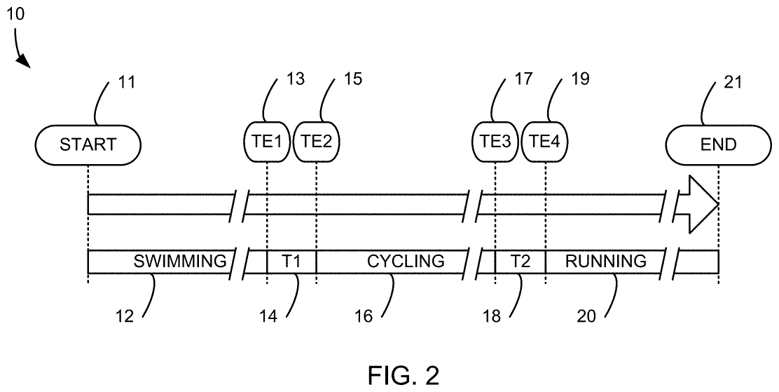

To provide context to the foregoing disclosure, FIG. 2 is a graphic illustration of an example triathlon event 10 and illustrates the different phases of a triathlon. As indicated, the triathlon event includes each of a swimming stage 12, a cycling stage 16, and a running stage 20 with the swimming stage 12 and the cycling stage 16 being separated by a first transition (commonly referred to as “T1”) 14 and the cycling stage 16 and the running stage 20 being separated by a second transition (commonly referred to as “T2”) 18. The triathlon also includes each of a start 11 and an end 21.

For purposes of the current disclosure, each segment of a given pair of concurrent segments of the triathlon event 10 is delineated by what is referred to herein as a transition event (“TE”). In general, a TE corresponds to any identifiable event indicating a change from one section or stage of the triathlon event 10 (e.g., the swim, bike, run, or transition stages) to a second, subsequent section of the triathlon event 10. More specifically the swimming stage 12 and T1 14 are delineated by a first TE (TE1) 13 at the end of the swim stage and beginning of the T1 stage. Similarly, T1 14 and the cycling stage 16 are separated by a second TE (TE2) 15 corresponding to the end of the T1 stage and the beginning of the cycling stage. The cycling stage 16 and T2 18 are separated by a third TE (TE3) 17 indicating the end of the cycling stage and beginning of the T2 stage. Finally, T2 18 and the running stage 20 are separated by a fourth TE (TE4) 19 indicating the end of the T2 stage and beginning of the run stage. In other words, each transition (e.g., the first transition between the swimming and cycling stages and the second transition between the cycling and running stages) is bounded by transition events signifying their respective beginning and ends.

The systems and methods described herein include approaches to identifying transition events between the various stages of a triathlon event and, based on identifying such transitions, to modify the operation of one or more exercise tracking devices. Although the current disclosure focuses primarily on triathlon applications, it should be appreciated that the described systems and methods are generally applicable to any event or race including multiple sections and, in particular, to such events or races that may include multiple sports. Accordingly, while the current disclosure focuses primarily on triathlons, the systems and methods discussed should not be seen as being limited to triathlon applications. For example and without limitation, aspects of the current disclosure may be adapted for use in duathlons (e.g., races including only two of the three conventional triathlon activities), ski biathlons (e.g., with transitions corresponding to shooting stages), adventure or expedition racing, long distance single activity racing (e.g., marathons), ski mountaineering racing, or any other similar event.

Example Operational Environment and Exercise Tracking Device

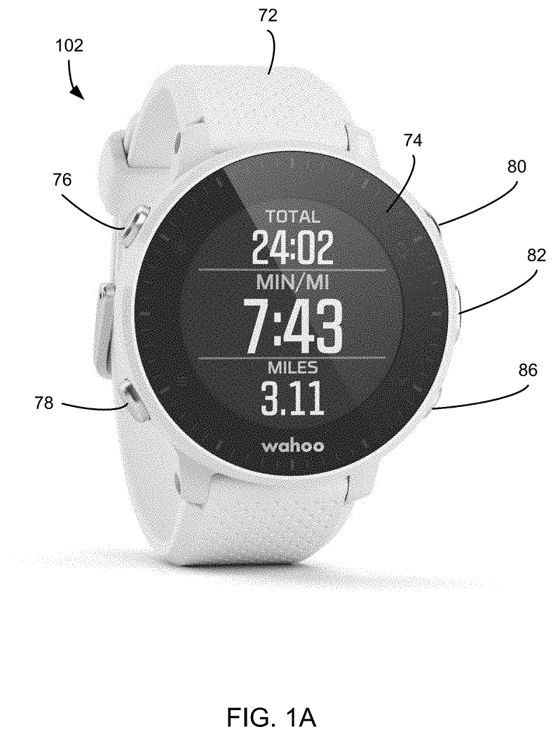

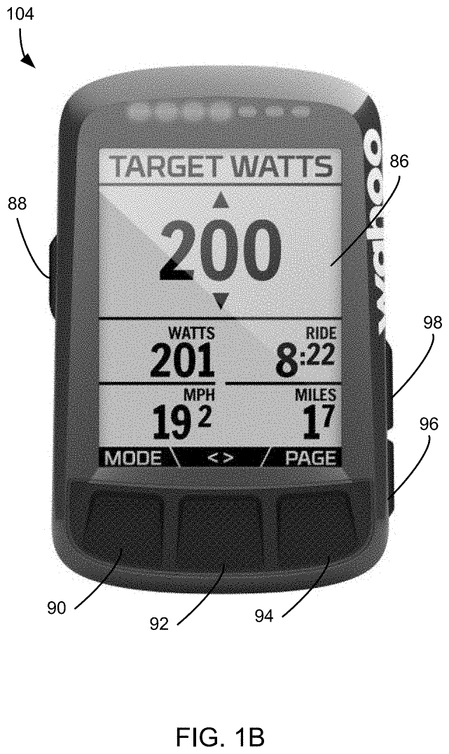

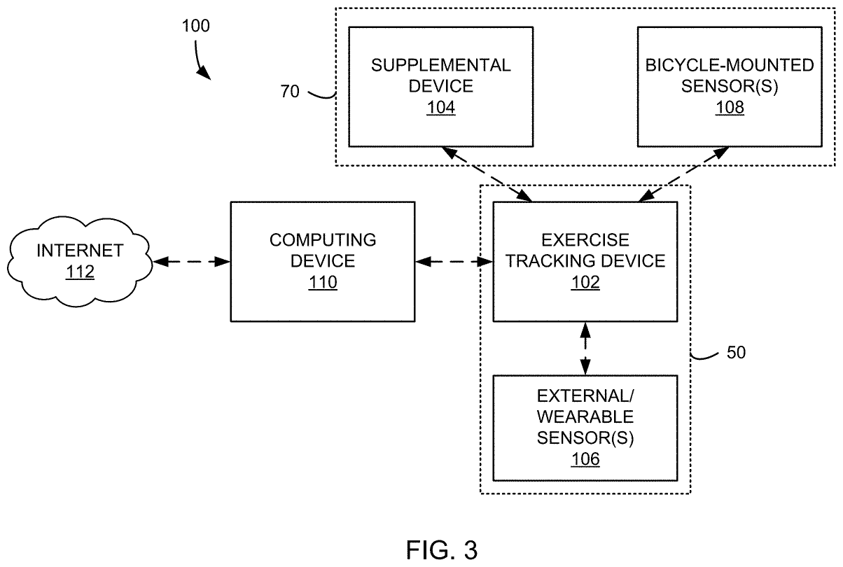

FIG. 3 is a schematic illustration of an operational environment 100 for tracking performance during a multi-sport event, such as the triathlon event 10 of FIG. 2. The operational environment 100 includes an exercise tracking device 102 (which may be, but is not limited to, a wrist-worn device, such as illustrated in FIG. 1A). Although other form factors may be implemented, in at least some applications the exercise tracking device 102 may be worn by a user 50 and may include, among other things, a wrist-mounted device, such as or similar to a smart watch. During operation, the exercise tracking device 102 captures and records fitness-related data from various sources and dynamically and automatically modifies its operational mode throughout the course of a multi-sport event, such as the triathlon event 10 of FIG. 2. With reference to the triathlon event 10, for example, the exercise tracking device 102 is generally configured to change operational modes in response to automatically detecting each TE.

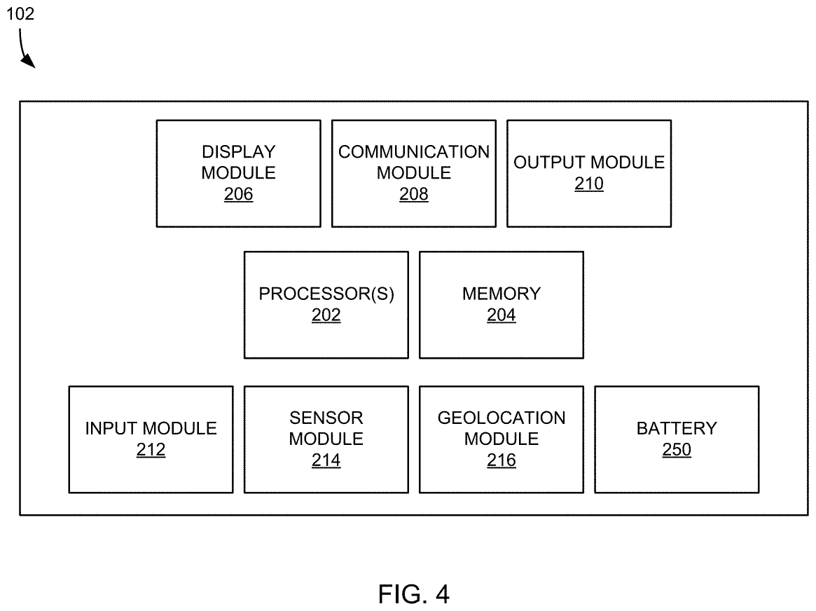

FIG. 1A is a rendering of an example exercise tracking device 102 in accordance with the present disclosure. As previously noted, the exercise tracking device 102 may generally be in the form of a wearable computing device. For example, the exercise tracking device 102 is illustrated in FIG. 1A in the form of a wrist-mounted watch including a watch band 72, a display 74 (which may be a touch-sensitive display) and various buttons 76-84 for controlling operations of the exercise tracking device 102. Additional features and functions of the exercise tracking device 102 are provided below and, in particular, in the context of FIG. 4, which is a block diagram illustrating the various components of the exercise tracking device 102.

Depending on the particular section of an event, the exercise tracking device 102 may be configured to collect and store data obtained from various internal and/or external sensors. For example, in one operational mode, the exercise tracking device 102 may be collect and store data from one or more internal sensors (as further described below in the context of FIG. 4). In another operational mode, the exercise tracking device 102 may be configured to receive data from one or more external/wearable sensors 106 such as, but not limited to, a wearable heartrate monitor. In yet another operational mode, the exercise tracking device 102 may collect and store data from one or more external sensors. Such external sensors may include bicycle-mounted sensors 108, such as one or more of a power meter, a cadence sensor, a speed sensor, or other similar sensor. The external sensors may also be part of a supplemental device 104, such as a computer or head unit mounted on a bicycle 70. For example, in one implementation, the exercise tracking device 102 is configured to operate in at least one mode in which the exercise tracking device 102 receives and stores geolocation data (e.g., global positioning system (GPS) coordinates) from a GPS module of the supplemental device 104. Conversely, the exercise tracking device 102 may be configured to operate in a mode in which the exercise tracking device 102 broadcasts data (e.g., heart rate or other sensor data) to the supplemental device 104. If the supplemental device 104 is tracking a current workout, the data sent by the exercise tracking device 102 is recorded by the supplemental device 104 as part of the workout.

FIG. 1B is a rendering of an example device that may be used as the supplemental device 104 for purposes of the present disclosure. More specifically, FIG. 1B illustrates an example cycling computer 104. As shown, the cycling computer 104 may include a display 86, which may, in certain implementations, be divided into multiple sections for simultaneously displaying data. The cycling computer 104 may further include inputs (such as buttons 88-98) for controlling various functions of the cycling computer 104.

As illustrated in FIG. 3, the exercise tracking device 102 may also be adapted to communicate with one or more computer devices 110. Such computing devices may include, without limitation, one or more of a smart phone, a tablet, a laptop computer, a desktop computer, or any other suitable computing device. In certain implementations, the computing device 110 may be configured to receive data from the exercise tracking device 102. The computing device 110 may then store the data, process the data, and/or transmit the data via the Internet 112 or similar network to one or more remote computing systems (not shown) for storage and/or processing. In some implementations, the supplemental device 104 is configured to communicate with sensor 108.

FIG. 4 is a block diagram of an example implementation of the exercise tracking device 102 of FIG. 3. As illustrated, the exercise tracking device 102 includes at least one processor 202 in communication with at least one memory 204. The exercise tracking device 102 further includes multiple modules in communication with and controllable by the processor 202, each of which is described below in further detail and each of which may be implemented as software, hardware, or a combination thereof. The exercise tracking device 102 further includes at least one battery 250 configured to power the exercise tracking device 102 and which may be any suitable type of battery. For example and without limitation, the battery 250 may include a rechargeable lithium-ion or lithium-polymer battery.

The exercise tracking device 102 may include a display module 206 configured to display information to a user of the exercise tracking device 102. In certain implementations, the display module 206 may include a liquid crystal display (LCD) or light-emitting diode (LED) screen that may be controlled or otherwise receive instructions from the processor to display different information and data. Such data and information may include, among other things, time metrics (e.g., total time, stage time, lap time), biometric information (e.g., heartrate metrics), speed and/or distance information, and navigation information. The display module 206 may also be used to present and navigate device-related information which may be presented in various menus or similar screens.

The exercise tracking device 102 may further include at least one communication modules 208 to facilitate communication between the exercise tracking device 102 and one or more external devices. Such external devices may include, for example, one or more of the supplemental device 104, the bicycle-mounted sensors 108, the wearable sensors 106, and the computing device 110 of the operational environment 100 of FIG. 3. The communication module 208 may be configured to communicate and support one or more communication protocols. Such protocols may include, without limitation, one or more of Bluetooth® (including Bluetooth® Low-Energy), ANT/ANT+, Wi-Fi, cellular, near-field communication (NFC), or any other similar communication protocols.

An output module 210 may be in communication with the processor 202 and may be configured to provide various forms of output to a user in response to instructions from the processor 202. In one example implementation, the output module 210 may include a speaker through which audible tones may be played to alert or otherwise notify a user of various events. As another example, the output module 210 may include a vibration motor or similar haptic device to generate vibration or other haptic outputs in response to various events.

The exercise tracking device 102 may also include an input module 212 adapted to receive one or more types of input from a user. Such inputs may then be processed by the processor 202 and used to initiate execution of instructions stored in the memory 204 or otherwise control operation of the exercise tracking device 102. The input module 212 may be configured to receive or include any of a wide range of inputs. In one implementation, the input module 212 may include one or more buttons, which may include physical buttons and/or “virtual” buttons presented on a display of the exercise tracking device 102. In the latter case, the input module 212 may include, at least in part, a touchscreen and, as a result, may be at least partially integrated with aspects of the display module 206. In other implementations, the input module 212 may additionally or alternatively include various other input mechanisms including, without limitation, one or more of a microphone, a haptic switch, a rotary dial, or any other similar mechanism.

The exercise tracking device 102 further includes at least one sensor module 214 that includes one or more sensors for measuring various parameters during use of the exercise tracking device 102. Although other sensors may be implemented in the exercise tracking device 102, in at least one implementation each of an accelerometer and a barometric pressure sensor may be included. The accelerometer may be configured to generally track and record movement of the exercise tracking device 102 and, as a result, may be used to interpret and analyze movement of the user of the device as described below in further detail. The barometric pressure sensor generally measures ambient pressure. Such ambient pressure measurements may be used to, among other things, determine a current elevation of the exercise tracking device 102 and/or determine whether the exercise tracking device 102 is submerged in water. In certain implementations, the sensor module 214 may also include a heartrate sensor, such as an optical heartrate sensor.

The exercise tracking device 102 may further include a geolocation module 216 for measuring a position of the exercise tracking device 102 and, as a result, a user of the device. In one implementation, the geolocation module 216 may be a global positioning system (GPS) module configured to periodically measure the current position of the exercise tracking device 102. The geolocation module 216 may be further configured to provide additional information based on the collected position information. For example and without limitation, such information may include speed information (e.g., current speed, average speed), and distance information (e.g., total distance travelled, distance travelled from a waypoint or similar location).

Example Operational Modes

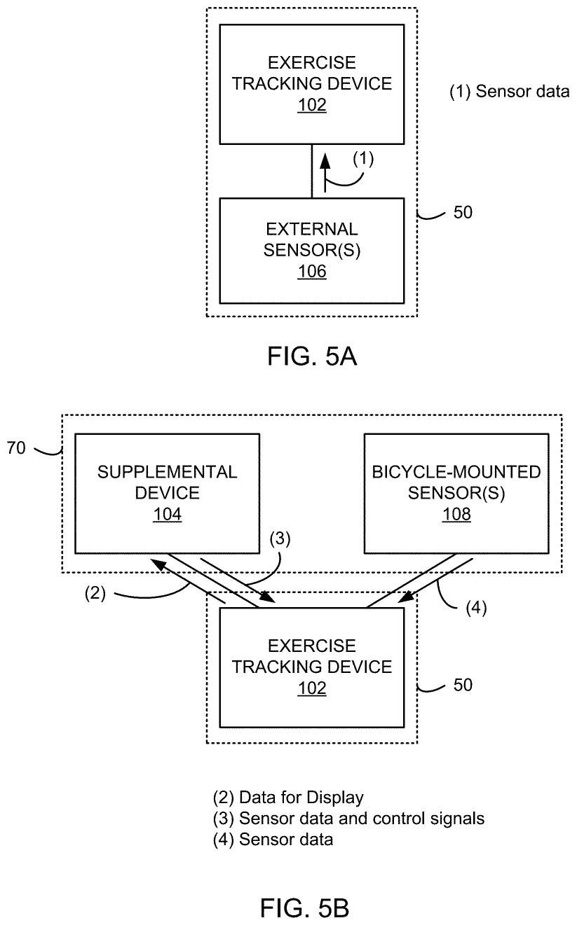

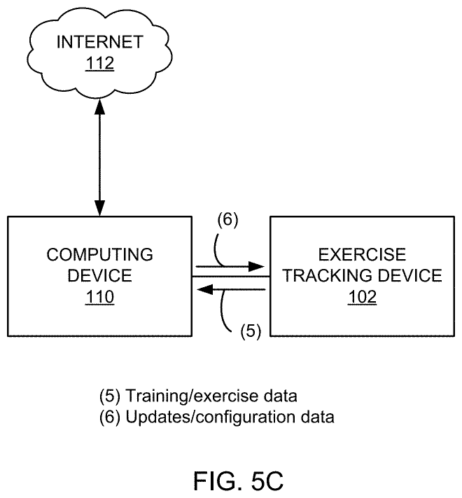

FIGS. 5A-5C illustrate various operational modes of the exercise tracking device 102. In general, the exercise tracking device 102 is configured to operate in various modes in which the exercise tracking device 102 receives data from or otherwise communicates with different devices, including different computing devices and different sensors. The exercise tracking device 102 is further configured to automatically transition between at least some of the operational modes during the course of an event and, in particular, in response to automatically detecting transition events, such as TE1-TE4 indicated in FIG. 2. Additional reference in the following discussion is made to the operational environment 100 of FIG. 3 and the block diagram of the exercise tracking device 102 of FIG. 4.

FIG. 5A is a block diagram of a first operational mode 400A in which the exercise tracking device 102 is configured to collect and store data from internal sensors and modules such as, but not limited to the input module 212, the sensor module 214, and the geolocation module 216 shown in FIG. 3. When in the first operational mode 400A, the exercise tracking device 102 may also be configured to receive and process data from one or more external/wearable sensors, such as but not limited to a heartrate monitor.

When in the first operational mode 400A, the exercise tracking device 102 is generally configured to perform geolocation-related functions using the geolocation module 216 of the exercise tracking device 102. As previously mentioned, such functions may include determining a current location of the exercise tracking device 102/user 50 and may further include calculating one or more additional metrics such as distance or speed.

In the context of a traditional triathlon event, the first operational mode 400A may generally correspond to a swimming or running mode. For example, when in the swimming mode, the exercise tracking device 102 may be configured to receive and process each of accelerometer and barometric pressure data from internal sensors and heartrate data from a wearable heartrate monitor communicatively coupled to the exercise tracking device 102 through the communication module 208. The accelerometer data may be used, for example, to detect movement of the user's arm and, based on such data, to calculate a stroke rate or similar cadence metric. The barometric pressure data may be used to determine if and when the exercise tracking device 102 is submerged and, as a result, if the user is still swimming. When in the running mode, the exercise tracking device 102 may operate using the same set of sensors, i.e., an internal accelerometer and an internal barometric pressure sensor with an external heartrate monitor, where the accelerometer may be used to determine running cadence and the barometric pressure sensor may be used to determine elevation.

Although illustrated in FIG. 5A as being coupled to an external/wearable sensor 106, it should be appreciated that in similar operational modes, the exercise tracking device 102 may operate using internal sensors only. For example, instead of an external heartrate monitor, the exercise tracking device 102 may instead rely on an internal heartrate monitor, such as an optical heartrate monitor, to track a user's heartrate.

FIG. 5B is a block diagram of a second operational mode 400B. In the second operational mode 400B, the exercise tracking device 102 is communicatively coupled to a supplemental device 104, such as a bicycle computer. The exercise tracking device 102 may further be communicatively coupled to one or more other sensors 108, which may include one or more bicycle-mounted sensors such as a power meter, a cadence sensor, or a speed sensor. When in the second operational mode 400B, the exercise tracking device 102 continues to store exercise-related data. Such exercise-related data may include data generated by internal sensors of the exercise tracking device 102, but may also include data from one or both of the supplemental device 104 and the one or more other sensors 108.

As illustrated in FIG. 5B, the exercise tracking device 102 may function as a “master” device to which each of the supplemental device 104 and the sensors 108 are connected. In such implementations, the exercise tracking device 102 is configured to communicate with each of the supplemental device 104 and the sensors 108 and to receive and store sensor data from them.

In certain implementations, each of the exercise tracking device 102 and the supplemental device 104 may have at least some duplicate functionality. For example, each of the exercise tracking device 102 and the supplemental device 104 may include a geolocation module, such as a GPS unit. As another example, each of the exercise tracking device 102 and the supplemental device 104 may include a display. In such implementations, when in the second operational mode 400B, at least some of such duplicate functionality of the exercise tracking device 102 may be disabled and handled exclusively by the supplemental device 104. So, for example, instead of relying on an internal GPS module to generate position data, the exercise tracking device 102 may instead receive such data from the supplemental device 104. Among other things, doing so reduces the demands on the exercise tracking device 102 and conserving battery life of the exercise tracking device 102 for tracking activity during stages in which the supplemental device 104 is not available.

In the context of a conventional triathlon, for example, the operational mode illustrated in FIG. 5B may correspond to a cycling mode. As illustrated in FIG. 2, the exercise tracking device 102 may enter the cycling mode upon identifying the transition event indicating the start of the cycling stage (e.g., TE2 15). Transitioning into the cycling mode may include, among other things, connecting to each of the supplemental device 104 and the bike-mounted sensor(s) 108. Transitioning into the cycling mode may also include disabling one or more modules of the exercise tracking device 102, such as the geolocation module 216 and/or the display module 206 illustrated in FIG. 4. Subsequent geolocation information may then be obtained from a corresponding geolocation module of the supplemental device 104 and communicated to the exercise tracking device 102. Similarly, any information that may normally be displayed on the exercise tracking device 102 may instead be provided to the supplemental device 104 for display. As a result, the display of the supplemental device 104 may effectively function as an additional or extended display of the exercise tracking device 102.

In certain implementations, the exercise tracking device 102 may store exercise data as a single time series. For example, a database or similar data structure may be maintained in the memory of the exercise tracking device 102. As exercise data is collected, entries may be periodically added to the data structure, each entry including a time stamp (e.g., a time stamp generated by an internal clock of the exercise tracking device) and values corresponding to one or more sensor measurements obtained from respective sensors of the exercise tracking device 102. When operating in the mode illustrated in FIG. 5B, the entries added to the data source may also or alternatively include sensor data obtained from one or more sensors or computing devices communicatively coupled to the exercise tracking device 102.

Using geolocation information as a non-limiting example, when the exercise computing device 102 is operated in the mode illustrated in FIG. 5A, the exercise computing device 102 populates the data source stored in the memory of the exercise computing device 102 with entries that include a time stamp and position data collected from a geolocation sensor of the exercise computing device 102. When the exercise computing device 102 begins operating in the mode illustrated in FIG. 5B, position information may instead be provided by the supplemental device 104. However, while the supplemental device 104 may provide the position information (and possibly related time information), the exercise computing device 102 generates and adds corresponding entries to the time series stored in the memory of the exercise computing device 102. Accordingly, while sensor data and other data may be obtained from sources external to the exercise tracking device 102, the exercise tracking device 102 is the single location of storing the data from both the exercise device and the supplemental device, and remains in control of any processing of such data.

As illustrated in FIG. 5B, data may be exchanged between the exercise tracking device 102 and the supplemental device 104 during operation. For example, in certain implementations, the exercise tracking device 102 may pass exercise, time, or other data for display on the supplemental device 104. Such data may include, but is not limited to summaries of previously recorded data (e.g., a summary of a swimming stage or T1 stage during a cycling stage) and current exercise statistics/metrics (e.g., distance travelled, speed metrics, heartrate, etc.).

In addition to data, the exercise tracking device 102 and the supplemental device 104 may be configured to exchange various control and input signals. For example, the supplemental device 104 may include a touchscreen or buttons that, when used, change the information displayed on a display of the supplemental device 104. In certain cases, such changes may include updating the display with information already stored within the supplemental device 104. In other cases, activating an input of the supplemental device 104 may cause the supplemental device 104 to transmit a message to the exercise tracking device 102 requesting certain information stored within or otherwise available from the exercise tracking device 102. Accordingly, in response to receiving such a message, the exercise tracking device 102 may generate and transmit a response message including the requested information to the supplemental device 104 for display by the supplemental device 104.

FIG. 5C illustrates a linked mode in which the exercise tracking device 102 is communicatively coupled to one or more other computing devices, such as computing device 110. When in the linked mode, the exercise tracking device 102 is capable of exchanging data with the computing device 110. For example, the exercise tracking device 102 may transmit training and exercise data collected and stored by the exercise tracking device 102 to the computing device 110. Similarly, the exercise tracking device 102 may receive configuration information, software updates, historical exercise and training data, training program information, and other similar data from the computing device 110.

In certain implementations, software executable on the computing device 110 may enable a user of the exercise tracking device 102 to review and analyze previously collected data and develop exercise and training programs. The computing device 110 may also allow for creation of routes for races or training events that may then be provided to the exercise tracking device 102. The exercise tracking device 102 may then use such information to guide a user during a race or workout. As discussed below in further detail, the computing device 110 may also be used to identify locations or areas corresponding to transitions between different stages of a particular event. Such transition areas may then be programmed into the exercise tracking device 102 to trigger transitions between different operational modes.

When in the linked mode, the computing device 110 may be in communication with the Internet 112 (or other network) and may be send and/or receive data via the Internet 112, such as to/from one or more cloud-based storage systems. Some or all of the previously discussed functions of the computing device 110 may also be performed by one or more servers accessible by the computing device 110 over the Internet 112. For example, the computing device 110 may include a web browser that enables a user to access a website or web portal that provides at least some of the functions of the computing device 110 described herein. Similarly, the computing device 110 may execute an app or other software that, while stored and executed locally on the computing device 110, may nevertheless communicate with one or more remote servers.

Although illustrated only in FIG. 5C as being communicatively coupled to the computing device 110 (i.e., only when in the linked mode), it should be appreciated that the exercise tracking device 102 may be connected to the computing device 110 in any of the previously described operational modes. For example, when in either of the swimming/running mode illustrated in FIG. 5A or the cycling mode illustrated in FIG. 5B, the exercise tracking device 102 may be communicatively coupled to a smart phone or similar computing device and may exchange data with the computing device.

Touchless Transitions

To facilitate tracking of exercise data during multi-sport events in which the exercise tracking device 102 operates in multiple modes, the exercise tracking device 102 may be configured to identify transitions and change operational modes in response to identifying the transitions. In certain implementations, such transitions occur automatically in response to some measured activity of the user meeting criteria indicating the occurrence of a transition event. In other words, the exercise tracking device 102 may be configured to change between operational modes without the user pressing a button or otherwise activating a similar input of the exercise tracking device 102. Accordingly, for purposes of the present disclosure, the process of automatically transitioning between operational modes is generally referred to as “touchless” transitioning. For purposes of the current disclosure, touchless transitioning may occur between one or more pairs of sequential stages (including transition stages) of a particular event. Touchless transitioning may also occur at one or both of the beginning of an event and the end of an event.

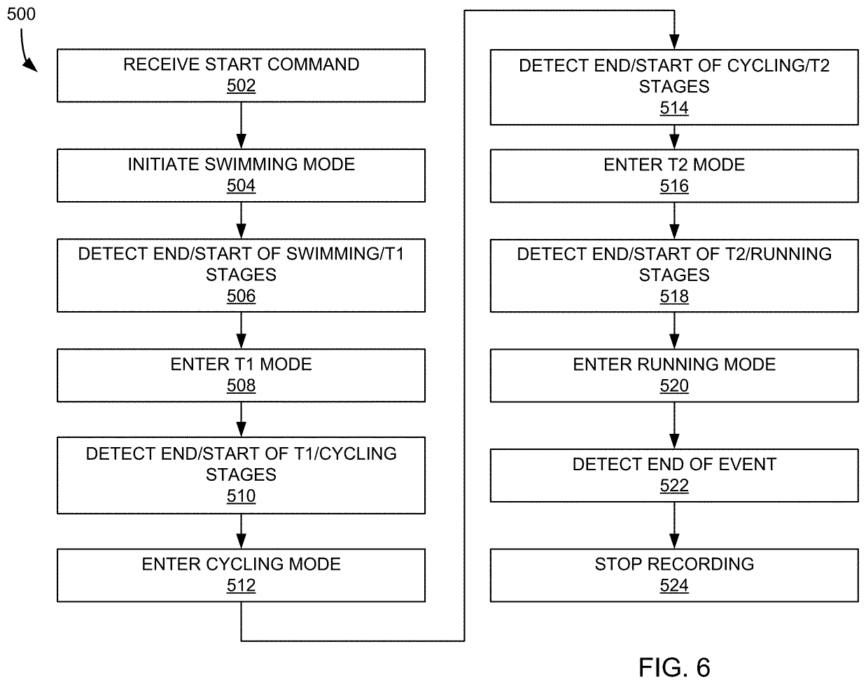

FIG. 6 is a flow chart illustrating a method 500 for transitioning between different operational modes during the course of a conventional triathlon event, such as illustrated in FIG. 2. As a result, the method 500 of FIG. 6 is provided in the context of an event including, in order, each of a swimming stage, a first transition stage (T1), a cycling stage, a second transition stage (T2), and a running stage. It should be appreciated that a conventional triathlon event is used only as an example and the method 500 may be adapted for use in other multi-sport or multi-stage events. In the following example, reference is also made to the various system and device components illustrated and described in the context of FIGS. 3 and 4. Such references should only be viewed as non-limiting examples.

At operation 502 the exercise tracking device 102 receives a start command indicating the beginning of the first stage of the event, which, for purposes of the current example is the swim mode. In certain implementations, the user may provide the start command by one or more inputs to the exercise tracking device 102, such as discussed in the context of the input module 212. For example and without limitation, inputs from the user may include a button press (including pressing a virtual button on a touch screen), a voice command, a haptic input (e.g., shaking the exercise tracking device 102), and the like.

In other implementations, the start command may be generated in response to data obtained from one or more sensors/modules of the exercise tracking device 102. Such data may include, among other things, measurements corresponding to movements obtained from an accelerometer (or similar sensor), changes in pressure or other environmental conditions, or changes in the location of the exercise tracking device 102. For example and without limitation, in implementations in which a swimming stage is the first stage, the start command may be correspond to one or more of a pressure reading from a barometric pressure sensor indicating the exercise tracking device 102 has been submerged or movements measured using an accelerometer indicating the user is performing a swimming stroke.

In another example, the start command may be provided in response to the user crossing a geo-fence or similar defined boundary. As described below in further detail, the exercise tracking device 102 may store geographic coordinates or similar information defining particular areas or boundaries. As the user enters/exits such an area or crosses such a boundary (e.g., as indicated by data received from the geolocation module 216), the exercise tracking device 102 may automatically generate a start command.

As other examples, the start command may be provided in response interactions between the exercise tracking device 102 and one or more external devices, such as another computing device, a transponder, a beacon, or similar device. For example, the starting line for an event may include a beacon or similar device configured to transmit a signal or otherwise communication with the exercise tracking device 102. The start command may then be automatically generated by the exercise tracking device 102 based on such communication. For example, the beacon may transmit a signal to the exercise tracking device 102 in response to the exercise tracking device 102 coming into range of/pairing with the beacon. In another example, the start command may be generated in response to communication between the exercise tracking device 102 and the other device being broken (e.g., in response to the beacon being deactivated or the exercise tracking device 102 being moved out of range of the other computing device).

In response to receiving the start signal, the exercise tracking device 102 enters a first operational mode, which, in the current example method 500, is a swim mode (operation 504). While in the swim mode, the exercise tracking device 102 may operate in a mode similar to that illustrated in FIG. 5A in which the exercise tracking device 102 functions primarily using internal sensors but may, in certain implementations, also pair with and receive data from one or more external sensors 106, such as a heart rate sensor. While in the swim mode, the exercise tracking device 102 may monitor and/or record sensor data including location data (e.g., from the geolocation module 216), heart rate data (e.g., from an internal heart rate sensor or as received from an external wearable sensor), movement data (e.g., as measured using an accelerometer and/or gyro), and pressure measurements (e.g., as measured using an internal barometric pressure sensor).

While in the swim mode, data collected by the exercise tracking device 102 may be tagged or otherwise grouped to identify it as data associated with the swimming stage. For example, in one implementation, data collected by the exercise tracking device 102 may be stored as a table or similar data structure within the exercise tracking device 102, with each entry of the data structure including an alphanumeric identifier associated with the swimming stage. In other implementations, a marker, tag, or similar entry may be inserted into or otherwise stored with the event data when the swim mode is initiated. Such a marker may include a timestamp indicating when the swim mode was initiated. Any subsequent data between the marker and a subsequent marker (e.g., indicating the end of the swimming stage or the beginning of a subsequent stage) may then be considered to be part of the swimming stage. Similarly, the initial portion of data collected after the exercise tracking device 102 receives the start command (e.g., operation 502) and before detecting any transitions may automatically be associated with a swim or similar first stage of an event

At operation 506, the exercise tracking device 102 detects a transition event indicating the end of the swimming stage and beginning of the first transition (T1) stage. Detecting the end of the swimming stage may be performed in various ways. However, in at least one implementation, the end of the swimming stage is detected in response to pressure measurements from a barometric pressure sensor of the exercise tracking device 102 indicating that the exercise tracking device 102 has not been submerged for a particular amount of time. In other implementations, detecting the end of the swimming stage may include detecting a change in movement patterns of the user. For example, the exercise tracking device 102 may determine that movement of the user does not correspond to a swim stroke for a particular period of time. In still other implementations, detecting the end of the swimming stage may also include detecting the exercise tracking device 102 crossing a geo-fence, as previously discussed in the context of generating the start signal or communicating or otherwise interacting with another computing device, such as a beacon or transponder, disposed at or near the end of the swimming stage.

Following detecting of the end of the swimming stage and start of the T1 stage, the exercise tracking device 102 may enter a T1 mode (operation 508). As previously discussed in the context of FIG. 2, in a conventional triathlon, the T1 stage generally corresponds to a transition between a swimming stage and a cycling stage. While in the T1 mode, the exercise tracking device 102 may continue to analyze and store measurements from one or more internal sensors of the exercise tracking device 102.

As part of operating in the T1 mode, the exercise tracking device 102 may also perform various processes associated with delineating between the swimming stage and the T1 stage. For example, the exercise tracking device 102 may stop a first timer associated with the swimming stage and start a second time associated with the T1 stage. The exercise tracking device 102 may also begin labelling collected data with a tag associated with the T1 stage, insert an entry indicating the start of the T1 stage in the collected data, or otherwise logging the transition between the stages. The exercise tracking device 102 may also perform one or more calculations on the data collected during the swimming stage to generate one or more summary metrics.

While operating in the T1 mode, the exercise tracking device 102 may also begin searching for and pairing with one or more devices. For example, using the communication module 208, the exercise tracking device 102 may begin to search for one or more devices or sensors that have been previously associated with the exercise tracking device 102. As previously discussed, such devices and sensors may include a supplemental device 104 (e.g., a cycling computer) and/or sensors 108, which may be coupled to a bicycle or other piece of equipment. Although various other approaches may be used, in at least one implementation, the exercise tracking device 102 may be paired with the one or more devices prior to the race with such pairing resulting in the exchange of identifying information. Subsequently, the identifying information may be broadcast by the exercise tracking device 102 or the one or more devices with the other device being configured to receive the identifying information and, in response to receiving the identifying information, to initiate a pairing/connection process.

At operation 510, the exercise tracking device 102 detects another transition event indicating the end of the T1 stage and beginning of a subsequent stage, which, in the current example, is a cycling stage. The process of detecting the beginning of the cycling stage may vary, however, in certain implementations, detecting the start of the cycling stage may include confirming pairing between the exercise tracking device 102 and a supplemental device 104 and/or other sensors 108 of a bicycle. In other implementations, detecting the start of the cycling stage may include receiving sensor data or other message from the supplemental device 104 and/or other sensors 108. For example, the cycling stage may be considered to have started when data from a power meter, speed sensor, cadence sensor, or other sensor is first received.

In still other implementations, the cycling stage may not be considered to have started until sensor data crosses a particular threshold. For example, the exercise tracking device 102 may be configured to detect the start of the cycling stage in response to determining the user is moving at or above a certain speed (e.g., 12 mph) indicative of cycling. Such sensor data may include sensor data from any of the exercise tracking device 102, the supplemental device 104, or any sensors 106, 108 that may be in communication with the exercise tracking device 102. In the specific case of speed, for example, speed measurements may be obtained from sensors of the bicycle (e.g., a bicycle-mounted speed sensor or GPS sensor) or from those of the exercise tracking device 102 (e.g., an accelerometer or GPS sensor).

Similar to previous stages, transition into the cycling stage may also be identified based on the user crossing a geo-fence and/or communication with one or more external devices, such as a beacon or transponder. The transition into the cycling stage may also be identified based on accelerometer or other measurements indicating movement of the user reflect that the user has started cycling.

In response to detecting the start of the cycling stage, the exercise tracking device 102 enters a cycling mode (operation 512). The cycling mode may generally correspond to the configuration in FIG. 5B in which the primary exercise device 102 receives and stores data provided by the supplemental device 104 and/or one or more bicycle-mounted sensors 108 and transmits data to the supplemental device 104 for display to the user. In some instances, the user also has the capability to manually transition between stages. For example, by interacting with an interface of the supplemental device 104 (i.e., bike computer), the user can change from a cycling state to a T2 state (or vice versa).

As previously discussed in the context of FIG. 5B, while in the cycling mode at least a portion of the functions handled by the exercise tracking device 102 in previous stages may instead by provided by the supplemental device 104. For example, the exercise tracking device 102 may transition into a low-power mode in which a display of the display module 206 and a geolocation sensor of the geolocation module 216 are disabled or otherwise operated in a reduced power mode. The display and geolocation functionality may then be provided by the supplemental device 104. More specifically, the supplemental device 104 may receive and display data from the exercise tracking device 102 and a GPS sensor (or similar geolocation sensor) of the supplemental device 104 may be used to collect location data that is then transmitted to and stored by the exercise tracking device 102. By doing so, battery life of the exercise tracking device 102 may be preserved for subsequent stages in which the exercise tracking device 102 is required to provide such functionality on its own. Although not illustrated in FIG. 5B, the exercise tracking device 102 may also still receive data from one or more other external sensors 106, such as a wearable heart monitor, when operating in the cycling mode.

At operation 514 the exercise tracking device 102 detects the end of the cycling stage and beginning of the second transition (T2) stage. Detecting the end of the cycling stage may be performed in various ways. However, in at least one implementation, the end of the cycling stage is detected in response to sensor measurements falling below a threshold. For example, one or more of measurements obtained from a speed sensor, a cadence sensor, a power meter, or other sensor may fall below respective thresholds indicating that a user is no longer riding the bicycle. In other implementations, detecting the end of the cycling stage may include detecting that movement patterns of the user no longer correspond to cycling. In still other implementations, detecting the end of the cycling stage may include detecting the exercise tracking device 102 crossing a geo-fence or otherwise interacting with another computing device, such as a beacon or transponder, disposed at or near the end of the cycling stage.

In certain implementations, detecting the end of the cycling stage and the start of the T2 stage may include reactivating one or more one or more modules or components that were previously disabled during the cycling stage. For example, as previously noted, each of a display and a GPS sensor (or other geolocation sensor) of the exercise tracking device 102 may be disabled during the cycling stage to conserve power. As a result, detecting the end of the cycling stage and start of the T2 stage may include reactivating the display and GPS sensor and confirming reactivation has been completed. With respect to the GPS sensor, confirming reactivation may include, among other things, receiving GPS data from the GPS sensor of the exercise tracking device 102.

Following detecting of the end of the cycling stage and start of the T2 stage, the exercise tracking device 102 may enter and operate in a T2 mode (operation 516). As previously discussed in the context of FIG. 2, in a conventional triathlon, the T2 stage generally corresponds to a transition between a cycling stage and a running stage.

When first entering the T2 stage, the exercise tracking device 102 may delineate the cycling data and subsequent data collected during the T2 stage, such as by beginning to label data using a tag associated with the T2 stage or inserting a marker or tag separating the cycling and T2 data. Upon entering the T2 mode the exercise tracking device 102 may also disconnect the various external devices and sensors used during the cycling stage, such as the supplemental device 104 and the bicycle-mounted sensor(s) 108. Alternatively, the exercise tracking device 102 may remain connected to but no longer record data from such devices and relying on the exercise tracking device 102 being moved out of range to trigger disconnection.

The exercise tracking device 102 may then detect the end of the T2 stage and start of another stage, which in the case of the current example is a running stage (operation 518). The transition between the T2 stage and the running stage may be detected in various ways. For example and without limitation, an accelerometer of the exercise tracking device 102 may detect movement of the exercise tracking device 102 indicative of running, a geolocation module of the exercise tracking device 102 may be used to determine the exercise tracking device 102 has crossed a geo-fence, location or accelerometer measurements may be used to determine that the exercise tracking device 102 is moving at a speed indicative of running, or the exercise tracking device 102 may transition in response to interaction with a nearby beacon or other device.

In response to detecting the start of the running stage, the exercise tracking device 102 may enter a running mode 520. Similar to the swimming mode described above in the context of operation 504, the running mode may generally include operating exercise tracking device 102 in a manner similar to that illustrated in FIG. 5A. More specifically, the exercise tracking device 102 operates substantially using its own internal sensors but may still receive data from one or more external sensors.

While in the running mode, the exercise tracking device 102 may track various running-related metrics. For example, the exercise tracking device 102 may use geolocation data to determine speed, position, elevation, and other location-based metrics. The exercise tracking device 102 may also track speed using an onboard accelerometer. The accelerometer may also be used to determine the user's cadence, among other things.

At operations 522 and 524, the exercise tracking device 102 detects the end of the event and stops recording, respectively. Similar to the transitions between stages, the end of the event may be detected based on various criteria. For example and without limitation, the end of an event may be determined based on geolocation data indicating that the user has crossed a geo-fence corresponding to a finish line or that the user's speed has dropped below a certain threshold indicating that the user has stopped running. Alternatively, the end of the event may be detected based on a change in the movement patterns of the user (e.g., a change from a running pattern to a walk or stationary pattern) as recorded using the accelerometer. In still another implementation, the user may provide an input (e.g., a button press) to the exercise tracking device 102, similar to the input used to indicate the beginning of the event in operation 502. In yet another implementation, the exercise tracking device 102 may detect the end of an event based on communication with a device disposed at or near the finish of the event.

After recording is completed, the user may access and review data through menus of the exercise tracking device 102. Alternatively or in addition to accessing the stored data using the exercise tracking device 102, the exercise tracking device 102 may be connected to and download data to another computing device (e.g., a smartphone, tablet, laptop, or desktop computer) as previously discussed in the context of FIG. 5C.

The method 500 illustrated in FIG. 6 and discussed above is merely one example of the implementation of touchless transitioning in a multi-stage event. Accordingly, the foregoing application may be modified to account for events having different stages (e.g., stages based on different sports or activities), different ordering of stages, or any other variations.

As previously discussed, at least certain transitions may be identified based on sensor data meeting a threshold value. For purposes of this disclosure, a threshold is met when sensor value is greater than a threshold value when the threshold value represents an upper limit or less than a threshold value when the threshold value represents a lower limit. Meeting the threshold in either or both cases may also include the sensor data being equal to the threshold value. In certain implementations, identifying a transition may require that a threshold be met for a particular time period.

The specific values to be used as thresholds may vary and may be particular to the specific transition to be identified. In certain implementations, for example, the threshold values may correspond to specific ranges of metrics that are indicative of certain activities. Speed thresholds, for example, may generally be used to identify when a user is swimming (relatively low speed, e.g., less than 5 mph), running (intermediate speed, e.g., between 5 mph and 10 mph), and cycling (relatively high speed, e.g., in excess of 10 mph). Similarly, pressure sensor data may be used to determine when a user is swimming or has exited the water. For example, a pressure threshold corresponding to atmospheric pressure may be implemented with a timer for determining the amount of time the exercise training device is at atmospheric pressure (e.g., out of the water) as opposed to a higher pressure when submerged. In certain implementations, the threshold values may be manually or automatically adjusted or set based on an individual users abilities and/or to otherwise tune when transitions are detected.

In some implementations, the system enables the user to revert from the current state to a prior state of their choosing. For illustration purposes, during a triathlon, the user may elect to transition from the running state back to the cycle state. In one example, the user presses the lap button on the exercise tracking device 102 which triggers a display of the different state. The user can in turn select the cycle state from the menu, thereby causing the state to change. Other techniques for allowing the user to change the state are also contemplated by this disclosure.

Power Management by Selective Activation/Deactivation of Device Modules

Multi-stage events, such as triathlons, present various power-related challenges for exercise tracking devices. In general, exercise tracking devices for such events must have sufficient battery life to continue operating and collecting data for the full duration of the event. However, increasing the size and or quantity of batteries for a given device increases the device's weight, negatively impacting the comfort of wearing and using the device. So, to meet battery life requirements without becoming overly cumbersome, conventional exercise tracking devices are often limited with respect to the quantity and/or type of sensors they may include. As a result of such limitations, the quantity, type, accuracy, and overall quality of data collected by conventional exercise tracking devices is often limited.

In light of the foregoing issues, implementations of exercise tracking devices in accordance with the present disclosure may include power saving functionality directed to improving overall battery life and data collection. In particular, exercise tracking devices described herein leverage the ability to pair with and receive data from one or more additional computing devices that a user may use during the course of a given event. When paired, at least a portion of the functionality provided by the exercise tracking device is instead offloaded to the additional computing device. As a result, the exercise tracking device may disable sensors or modules corresponding to such functionality when paired. Alternatively the exercise tracking device may instead operate the sensors or modules in reduced power modes. In either case, the exercise tracking device conserves power for later use when the exercise tracking device is not paired with an additional computing device.

One example application of the power management techniques described herein is in the context of a triathlon event. During the swimming stage, the exercise tracking device may operate independently using a first set of sensors and/or modules. At the beginning of the cycling stage, the exercise tracking device may pair with a cycling computer and offload at least some of the functions associated with the first set of sensors/modules to the cycling computer. In one specific example, geolocation data may be collected by a geolocation module of the cycling computer and transmitted to the exercise tracking device for storage. Similarly, any data previously displayed on a display of the exercise tracking device may instead be displayed on a screen of the cycling computer. Accordingly, each of the geolocation and display modules of the exercise tracking device may be disabled or operated in a reduced power mode during the cycling stage. When the user completes the cycling stage, the exercise tracking device may disconnect from the cycling computer and enable each of the geolocation and display modules for use during the running stage.

While the geolocation and display modules of the exercise tracking device are examples of relatively high power consumption components, the approach to selectively activating and deactivating components of the exercise tracking device described herein are not limited to geolocation and display modules. Rather, any sensor or module of the exercise tracking device for which corresponding data may instead be collected and provided by a supplemental device may be subject to deactivation in response to connecting to and receiving the corresponding data from the supplemental device. Provided that the power required to connect to and process data received from the supplemental device is less than would be required to collect and process data from sensors/modules of the exercise tracking device, a net power savings is realized. It should also be appreciated that the sensor/module of the supplemental device does not necessarily have to be the exact same type of sensor/module as the disabled sensor/module of the exercise tracking device. Rather, the module of the supplemental device need only provide the same type of data as the disabled module of the exercise tracking device. For example,

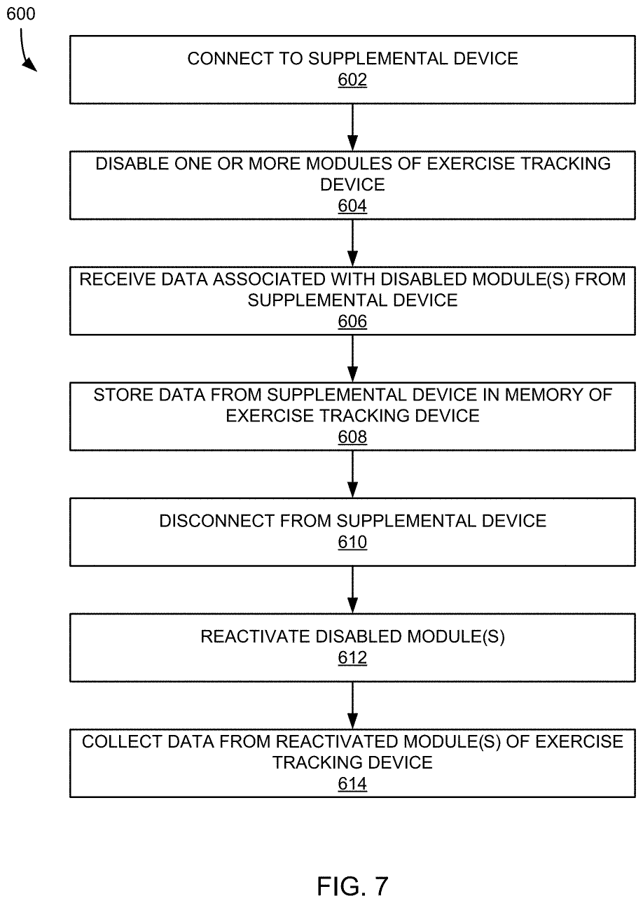

FIG. 7 is a flow chart illustrating a method 600 of power management in accordance with the present disclosure. Reference is made in the following discussion to the various components of FIGS. 3-5C and various steps of the method 500 described in FIG. 6. Such references are intended merely to provide context and should not be viewed as limiting the following description to the specific implementations illustrated in FIGS. 3-6.

At operations 602, the exercise tracking device 102 connects with a supplemental device 104. Once connected, the exercise tracking device 102 disables one or more sensors/modules (operation 604). The process of connecting to the supplemental device and disabling modules of the exercise tracking device 102 may occur, for example, as part of the processing of detecting the end of the T1 stage (and beginning of the cycling stage) and entering a cycling mode, as indicated in operations 510 and 512 of the method 500 of FIG. 6.

As previously noted, the particular modules of the exercise tracking device 102 disabled during operation 604 may vary based on the capabilities of the supplemental device 104. However, in at least one implementation, the modules disabled during operation 604 may include at least one of a geolocation module and a display module. For purposes of the present discussion, the term disable should be construed to include any of complete deactivation of a module, partial deactivation of a module (e.g., deactivating one or more components or submodules of a given module), or modifying operation of a module (in whole or in part) such that the module operates in a reduced power mode and provides a reduced set of functions.

At operations 606 and 608, the exercise tracking device 102 receives and stores data from the supplemental device 104, respectively. More specifically, the exercise tracking device 102 receives data from the supplemental device 104 that would have otherwise been provided by the modules disabled during operation 604.

At operation 610, the exercise tracking device 102 is disconnected from the supplemental device 104. Referring back to FIG. 6, such disconnection may occur during the process of detecting the end of the cycling stage/beginning of the T2 stage and entering the T2 mode, as included in operations 514 and 516, respectively.

In response to or as part of disconnecting from the supplemental device 104, the exercise tracking device 102 reactivates the modules previously disabled in operation 604. The exercise tracking device 102 may then begin collecting and storing data from the reactivated modules (operation 614).

Use of Geofencing for Transitions

As previously noted in the context of FIG. 6, transitions between stages of given event may be facilitated, at least in part, by implementing geofencing functionality. More specifically, in at least one implementation of the present disclosure, one or more of the transitions between stages may be detected when the exercise tracking device crosses over a virtual geographic boundary. In response, the exercise tracking device may change operational modes.

Geofencing may be implemented to detect the transition between any stages of a given event. For example, one or more geofences may be defined corresponding to transition areas between stages of a given event. The exercise tracking device may then be configured to change operational modes in response to one or more of entering or exiting the boundary defined by the geofence. In one specific example, the exercise tracking device changes from a first operational mode (e.g., the swimming mode) to a second operational mode (e.g., the T1 mode) as the user enters the geofence corresponding to a transition area. As the user exits the transition area, the exercise tracking device may change from the second operational mode to a third operational mode (e.g., a cycling mode).

For the purpose of the following discussion, the term operational mode is used to describe any operational state of the exercise tracking device. In one implementation, different operational modes may correspond to different sets of sensors/modules of the exercise tracking device being activate (e.g., as previously discussed in the context of FIG. 7). However, different operational modes may also correspond to different ways in which the exercise tracking device processes and/or stores data or changes in the data output by the exercise tracking device. Accordingly, while geofencing may be used to trigger changes between operational modes corresponding to stages of a given event, the term operational mode should be understood to more broadly refer to any change in operation of the exercise tracking device.

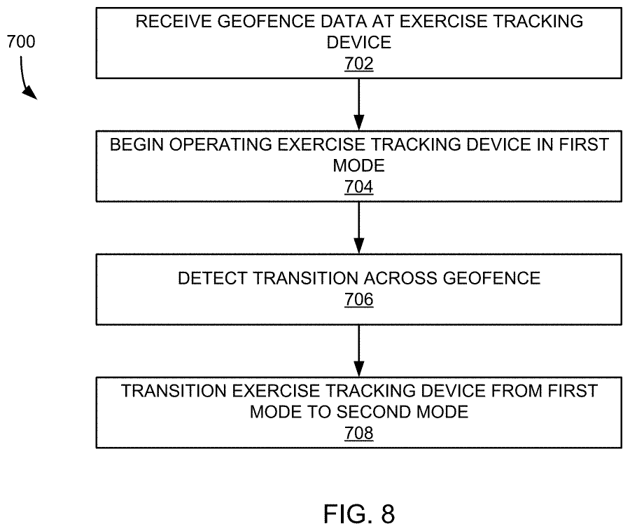

FIG. 8 is a flow chart illustrating an example method 700 for using geofencing in implementations of the present disclosure. In general and as described below in further detail, the process of implementing geofencing includes receiving data defining the geofence. The position of the exercise tracking device is then monitored to determine when the exercise tracking device crosses the virtual boundary. In response to crossing the boundary, the exercise tracking device automatically changes operational modes.

At operation 702, the exercise tracking device 102 receives geofence data. For example, the exercise tracking device 102 may receive a set of geographic coordinates defining a geographic area, the perimeter of which forms the geofence. Accordingly, in certain implementations, the exercise tracking device 102 may be able to interpolate between individual geographic coordinates.

The geofence data may be received in various ways. In one implementation, the exercise tracking device 102 may receive the geofence data from a computing device to which the exercise tracking device 102 may be connected. For example, the computing device may be used to access software or a website from which geofence data may be downloaded. In one particular application, a website may include a link from which predefined geofence data corresponding to key geographic areas (e.g., start/finish lines, transition areas) of a given event may be retrieved. In other applications, a map or similar graphical user interface may be provided to a user of the computing device. The user may then define the geofence using the interface, such as by providing a list of geographic points or drawing points or polygons on the presented map corresponding to the geofence. Data corresponding to the user-created geofence may then be downloaded and stored within the exercise tracking device 102.

In another implementation, the exercise tracking device 102 may allow a user to generate the geofence data by recording the current location of the exercise tracking device 102. For example, the exercise tracking device 102 may be operable in a geofencing mode in which a user stands at locations corresponding to points of the geofence while wearing or holding the exercise tracking device 102. The exercise tracking device 102 may then store its current location (e.g., in response to a button press by the user or in response to detecting the exercise tracking device 102 is substantially stationary) as a point of geofence data. In another implementation, the exercise tracking device 102 may be operable in an alternative geofencing mode in which a user may walk along the perimeter of the geofence while wearing or holding the exercise tracking device 102 and the exercise tracking device 102 periodically samples its current location, each sample forming a point of the geofence data.

At operation 704 the exercise tracking device 102 begins operation in a first mode and continues to operate in the first mode until movement across the geofence is detected (operation 706). In general, detecting movement across the geofence includes receiving current locations of the exercise tracking device 102 and comparing the current location with the stored geofence data. If the current locations indicate that the exercise tracking device 102 was previously within the geofence and has since exited its perimeter (or was previously outside the geofence and has since entered the geofence), the exercise tracking device 102 is considered to have crossed the geofence. In response to detecting such movement across the geofence, the exercise tracking device 102 may change from the first operational mode to a second operational mode (operation 708).

Although the foregoing example refers to the location of the exercise tracking device 102 and determining when the exercise tracking device 102 crosses the geofence, it should be appreciated that the location of a supplemental device (such as supplemental device 104) may instead be used to approximate or as a proxy for the location of the exercise tracking device 102. For example, when operating in a mode in which power is conserved by offloading geolocation functionality to the supplemental device 104 (e.g., the cycling mode discussed in the context of operation 512 of FIG. 6), the location data used to determine whether a geofence has been crossed may actually correspond to the location of the supplemental device 104 and not that of the exercise tracking device 102. Nevertheless, due to the relatively close proximity within which the exercise tracking device 102 and the supplemental device 104 are maintained, detecting crossing of a geofence based on the location of the supplemental device 104 may be sufficient to indicate that the exercise tracking device 102 has similarly crossed the geofence.

The techniques described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

Some portions of the above description present the techniques described herein in terms of algorithms and symbolic representations of operations on information. These algorithmic descriptions and representations are the means used by those skilled in the data processing arts to most effectively convey the substance of their work to others skilled in the art. These operations, while described functionally or logically, are understood to be implemented by computer programs. Furthermore, it has also proven convenient at times to refer to these arrangements of operations as modules or by functional names, without loss of generality.

Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the described techniques include process steps and instructions described herein in the form of an algorithm. It should be noted that the described process steps and instructions could be embodied in software, firmware or hardware, and when embodied in software, could be downloaded to reside on and be operated from different platforms used by real time network operating systems.

The present disclosure also relates to an apparatus for performing the operations herein. This apparatus may be specially constructed for the required purposes, or it may comprise a computer selectively activated or reconfigured by a computer program stored on a computer readable medium that can be accessed by the computer. Such a computer program may be stored in a tangible computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The algorithms and operations presented herein are not inherently related to any particular computer or other apparatus. Various systems may also be used with programs in accordance with the teachings herein, or it may prove convenient to construct more specialized apparatuses to perform the required method steps. The required structure for a variety of these systems will be apparent to those of skill in the art, along with equivalent variations. In addition, the present disclosure is not described with reference to any particular programming language. It is appreciated that a variety of programming languages may be used to implement the teachings of the present disclosure as described herein.

Although various representative embodiments have been described above with a certain degree of particularity, those skilled in the art could make numerous alterations to the disclosed embodiments without departing from the spirit or scope of the inventive subject matter set forth in the specification. All directional references (e.g., upper, lower, upward, downward, left, right, leftward, rightward, top, bottom, above, below, vertical, horizontal, clockwise, and counterclockwise) are only used for identification purposes to aid the reader's understanding of the embodiments of the present invention, and do not create limitations, particularly as to the position, orientation, or use of the invention unless specifically set forth in the claims. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

In methodologies directly or indirectly set forth herein, various steps and operations are described in one possible order of operation, but those skilled in the art will recognize that steps and operations may be rearranged, replaced, or eliminated without necessarily departing from the spirit and scope of the present invention. It is intended that all matter contained in the above description or shown in the accompanying drawings shall be interpreted as illustrative only and not limiting. Changes in detail or structure may be made without departing from the spirit of the invention as defined in the appended claims.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1.-8. (canceled)

9. A method of changing operational modes of exercise tracking devices comprising:
obtaining, at an exercise tracking device operating in a first mode, location data for the exercise tracking device;
comparing the location data to geographic data stored in a memory of the exercise tracking device, the geographic data defining a geofence; and
in response to determining the exercise tracking device has crossed the geofence, transitioning the computing device from the first mode of operation to a second mode of operation.

10. The method of claim 9 further comprising storing the geographic data defining the geofence in the memory of the exercise tracking device.

11. The method of claim 10 further comprising obtaining each of a plurality of geographic data points of the geographic data by, for each geographic data point:
obtaining current location data of the exercise tracking device using a geolocation sensor; and
storing at least a portion of the current location data as the geographic data point.

12. The method of claim 10 further comprising:
accessing the geographic data from a remote computing device; and
downloading the geographic data to the memory of the exercise tracking device.

13. The method of claim 9 further comprising obtaining the location data from a second computing device communicatively coupled to the exercise tracking device.

14. The method of claim 13, wherein the secondary device is a cycling computer.

15. The method of claim 9, wherein the location data is obtained from a geolocation sensor of the exercise tracking device.

16. The method of claim 9, further comprising:
obtaining, in the first mode of operation, location data from a first of the exercise tracking device itself and a second computing device communicatively coupled to the exercise tracking device; and
obtaining, in the second mode of operation, location data from a second of the exercise tracking device itself and the second computing device.

17. A method of tracking exercise data for a multi-stage activity, the method comprising:
collecting, using a wearable computing device, a plurality of sensor data streams,
automatically identifying a plurality of transitions between stages of the multi-stage activity; and
in response to identifying each of the plurality of transitions, automatically changing the computing device between operational modes, each operational mode corresponding to a respective stage of the multi-stage event,
wherein the wearable computing device records exercise-related data corresponding to the plurality of sensor data streams during each of the operational modes, and, in a subset of the operational modes, the wearable computing device is communicatively coupled to a secondary device and receives at least one of the sensor data streams from the secondary device.

18. The method of claim 17, wherein automatically identifying at least one transition of the plurality of transitions comprises determining at least one of the wearable computing device being communicatively coupled with the secondary device and being communicatively decoupled from the secondary device.

19. The method of claim 17, wherein automatically identifying at least one transition of the plurality of transitions comprises determining at least one of the plurality of sensor data streams:
changes in value;
meets a threshold value or meets the threshold value for a particular time;
indicates a change of a movement pattern of a user of the wearable computing device;
indicates a user of the wearable computing device is travelling above a speed threshold;
indicates a user of the wearable computing device is travelling below a speed threshold; and
indicates a user of the wearable computing device has crossed a geofence.

20. The method of claim 17, wherein when the wearable computing device is communicatively coupled to the secondary device and receives at least one of the sensor data streams from the secondary device, operating a corresponding sensor of the wearable computing device capable of providing the at least one sensor data stream in a reduced power mode.

21. The method of claim 17 further comprises receiving, via a user interface on the wearable computing device, an input from a user of the wearable computing device and, in response to receiving the input, changing a current operational mode of the wearable computing device to another operational mode.

22. The method of claim 21 wherein the stages of the multi-stage event occur in a predefined sequence and the another operational mode corresponds to a stage that precedes the stage that corresponds to the current operational mode in the predefined sequence.

23. The method of claim 17 further comprises
broadcasting one or more of the sensor data streams in the plurality of sensor data streams from the wearable computing device to the secondary device; and
recording the broadcasted sensor data streams in a memory of the secondary device.
